(19) United States
(12) Patent Application Publication
(10) Pub. No.: US 2019/0054253 A1
(43) Pub. Date: Feb. 21, 2019

(54) IMPLANT SYRINGE

(71) Applicant: Gaplast GmbH, Altenau (DE)

(72) Inventors: Stephan Kneer, Farchant (DE); Roland Kneer, Farchant (DE)

(73) Assignee: Gaplast GmbH, Altenau (DE)

(21) Appl. No.: 16/103,960

(22) Filed: Aug. 16, 2018

(30) Foreign Application Priority Data
Aug. 19, 2017 (DE) 10 2017 007893.9

(51) Int. Cl. A61M 5/32; A61M 5/315

(52) U.S. Cl. CPC A61M 5/3204; A61M 2005/3143; A61M 5/31515

(57) ABSTRACT

An implant syringe with a syringe needle holder, having an axially adjoining preparation receptacle; an outer sleeve with a radially projecting front gripping section, connected together for common axial movement; an inner sleeve, on which the outer sleeve is slidably located and in which the syringe needle is movable with the preparation receptacle. The inner sleeve has axial slots, through which webs engage, which connect the outer sleeve to the syringe needle holder, further including a rod-shaped piston, fastened to a rear gripping section partially surrounded by a rear sleeve, movably engaged in the inner sleeve. The rod-shaped piston is advanceable through the preparation receptacle in the syringe needle so that a distance remains between the tip of the syringe needle and the head end of the piston approximately equal to the length of the preparation to be delivered.

### PRIORITY CLAIM

This application claims priority under 35 U.S.C. §§ 119 and 365 to German Application No. 10 2017 007893.9, filed Aug. 19, 2017

### BACKGROUND OF THE INVENTION

The invention relates to an implant syringe, which is used to deliver a thread-like preparation with a long-term active substance into the body of a patient. The long-term preparation is generally positioned in the abdominal wall of a patient, into which a receiving passage for the preparation was previously pierced by means of a syringe needle.

DE 19734385 C1 discloses an implant syringe, which includes a syringe needle arrangement consisting of a syringe needle, a projecting gripping member and a preparation receptacle, which are connected together and are situated in a guide device for common axial movement. The implant syringe further has a piston, whose advance in the direction of the syringe needle is limited by an abutment so that a distance remains between the tip of the injection needle and the head end of the piston, which is at least equal to the length of a preparation to be delivered, whereby the syringe needle arrangement is retractable by means of the gripping member by at least a distance equal to the length of the preparation. This implant syringe is operated so that firstly the insertion passage is formed by the syringe needle being pierced, for instance into the abdominal wall of a patient until the front edge of a guide sleeve of the implant syringe rests on the skin of the patient. The piston is then advanced by engaging behind a base plate of a spacer on the implant syringe and exerting pressure on an end plate connected to the piston. The piston pushes the preparation forward into the syringe needle until a sleeve connected to the piston impinges against the base plate of the spacer. The implant syringe is then gripped at the front gripping member and the base plate of the spacer, whereafter the gripping member is retracted to an abutment. On impingement against the abutment, the syringe needle is completely retracted from the preparation, which is thus delivered into the insertion passage, whereafter the implant syringe is removed. A preparation may be delivered gently into the insertion passage with such an implant syringe. The disadvantage of this known implant syringe resides in the fact that in order to retract the syringe needle it is necessary to engage around it, whereby the manipulation of the syringe is rendered difficult.

DE 19961197 B4 discloses an implant syringe in which this disadvantage is avoided. In this implant syringe, the gripping position need not be altered. The advance of the piston is followed directly by the retraction of the syringe needle so that its handling is facilitated and requires a lesser period of time. Disadvantages of this implant syringe are seen in the fact that the gripping member, with which the syringe needle arrangement is retracted, is arranged on the front end region of the implant syringe so that when operating the implant syringe the fingers of the hand of the doctor must be spread widely and that after retracting the syringe needle there is the risk of injuring oneself on the syringe needle due to the slotted guide sleeve. Since furthermore the preparation is advanced by the piston through a constriction in the passage, the preparation can be abraded or even break.

### SUMMARY OF THE INVENTION

It is the object of the present invention to provide an implant syringe in which at least one of the above disadvantages is avoided.

The object is solved in accordance by providing an implant syringe comprising a syringe needle holder, which holds an injection needle, an axially adjoining preparation receptacle and an outer sleeve with a radially projecting front gripping section on its rear end section. The injection needle holder and the outer sleeve are connected together for common axial movement, including an inner sleeve, on which the outer sleeve is slidably located and in which the syringe needle is movable with the preparation receptacle, The inner sleeve has axial slots, through which webs extend, which connect the outer sleeve to the syringe needle holder. The syringe further comprises a rod-shaped piston, which is fastened to a rear gripping section and is partially surrounded by a rear sleeve, which movably engages in the inner sleeve. The rod-shaped piston is advanceable through the preparation receptacle into the syringe needle so far that a spacing remains between the tip of the syringe needle and the head end of the piston, which is approximately equal to the length of the preparation to be delivered. The advance of the piston is limited by the rear gripping section reaching the rear edge of the inner sleeve. The outer sleeve is releasably locked in the advanced starting position with the syringe needle exposed by a blocking device, which is arranged on the inner sleeve and projects radially outwards. The blocking device is moved radially inwards on advance of the rear sleeve into its end position, whereby the outer sleeve is released and is retractable by means of the front gripping section on the inner sleeve.

Advantageous embodiments of the invention are described herein below.

The implant syringe in accordance with the invention has a syringe needle holder, to which a syringe needle is fastened, adjoining which there is a preparation receptacle, the passage of which merges into the passage of the syringe needle. The syringe needle holder is connected to an outer sleeve of the implant syringe, which has a projecting gripping section at its rear end, which is referred to as the front gripping section, whereby the syringe needle, the syringe needle holder and the preparation receptacle are connected to the outer sleeve for common axial movement together.

The outer sleeve is located slidably on an inner sleeve, whereby the outer sleeve is non-rotatably arranged with respect to the inner sleeve. In an intermediate region, the inner sleeve has a plurality of slots, which are spaced from one another in the peripheral direction and through which webs engage, which connect the outer sleeve to the syringe needle holder. The slots extend preferably over a length which is the same as the range of movement of the syringe needle holder.

The implant syringe in accordance with the invention further includes a rod-shaped piston, which is fastened to a preferably circular plate, which serves as a rear gripping section on actuation of the implant syringe. The piston is surrounded over a rear portion of its length by a sleeve, which engages slidably and non-rotatably in the inner sleeve. The piston may be advanced so far through the preparation receptacle into the syringe needle that a spacing remains between the tip of the syringe needle and the head end of the piston, which is approximately equal to the length of the of the preparation to be delivered or is somewhat larger. The advance of the piston is limited by the rear gripping section reaching the rear edge of the inner sleeve. Until this occurs, the outer sleeve is locked in its advanced starting position, in which the syringe needle is exposed, by a blocking device so that the outer sleeve cannot be retracted with the syringe needle. The blocking device is preferably formed on the inner sleeve and projects radially beyond it outwardly so that it blocks the retraction of the outer sleeve. In this position, it can engage, for instance, a rear shoulder on the outer sleeve. When the rear sleeve is advanced into its end position, the blocking device is pulled radially inwardly so that the path of movement of the outer sleeve is free and it can be retracted together with the syringe needle by means of the front gripping member.

In this construction, the distance between the two gripping members, which are gripped to operate the implant syringe, is considerably reduced since the front gripping member is situated at the end of the elongate outer sleeve. The operation of the implant syringe is hereby made considerably easier, particularly for those people who have small hands.

It is proposed with great advantage that the blocking device is a spring arm cut free from the inner sleeve, the locking hook of which projects radially beyond the inner sleeve in the condition in which it is unloaded by the rear sleeve and blocks the retraction of the outer sleeve. The rear sleeve can have an axial slot, into which the spring arm slides, seen relatively, wherein at least one oblique surface impinges against a lateral formation on the spring arm or impinges directly on the spring arm, which pulls the spring arm radially inwards and thus removes the locking hook from the path of movement of the outer sleeve.

This process is complete when the rear sleeve has reached its front end position, in which the rear gripping section abuts against the rear edge of the inner sleeve.

The retraction of the outer sleeve is preferably terminated when the two gripping sections engage one another. It is preferred that when the outer sleeve is completely retracted the piston projects beyond the tip of the syringe needle.

In a further advantageous embodiment of the invention, a spring arm projects into the passage in the preparation receptacle, which rests on the preparation and fixes it in position or constricts the passage in the unloaded state so that movement of the preparation is blocked so that the preparation cannot discharge out of the implant syringe when this is not desired. It is advantageously provided that the spring arm projects flatly into the passage, preferably flatly convexly curved. When the piston advances the preparation through this constriction, the spring arm is pressed outwardly, whereby this process occurs so gently that the preparation is not abraded or cannot break. It is also proposed with advantage that the inner sleeve has a head section adjoining the slotted region, which is closed over its periphery and which covers the tip of the syringe needle in its retracted end position. The head section has a frustoconical shape which preferably tapers forwardly. It is thus ensured that even when the implant syringe removed from the patient is handled in an unskilled manner there is no risk of injury by the tip of the syringe needle.

Further details of the invention will be apparent from the following description of a preferred embodiment of the implant syringe and with reference to the drawings, in which, to different scales.

### BRIEF DESCRIPTION OF THE DRAWINGS

FIGS. 1a to 1c are different views of an embodiment of the implant syringe;

FIGS. 2a to 2d are different views of the rear sleeve with the rod-shaped piston;

FIGS. 3a to 3d show further details of the rear sleeve;

FIG. 4 is a view of the inner sleeve composed of two parts;

FIGS. 5a to 5d are different views of the front portion of the inner sleeve;

FIGS. 6a to 6d are different views of the rear portion of the inner sleeve;

FIG. 7 is a longitudinal sectional view of the outer sleeve;

FIGS. 8a to 8d are different views of the preparation receptacle;

FIG. 9 shows the blocking device of the outer sleeve.

### DETAILED DESCRIPTION

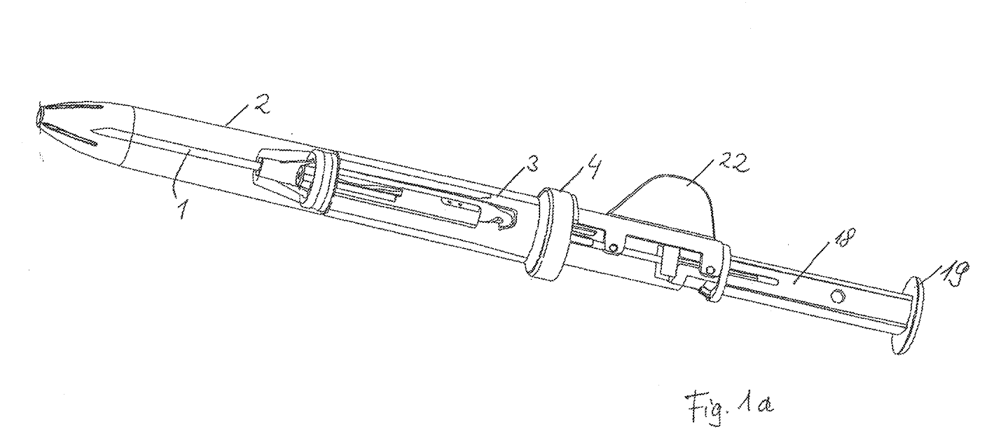

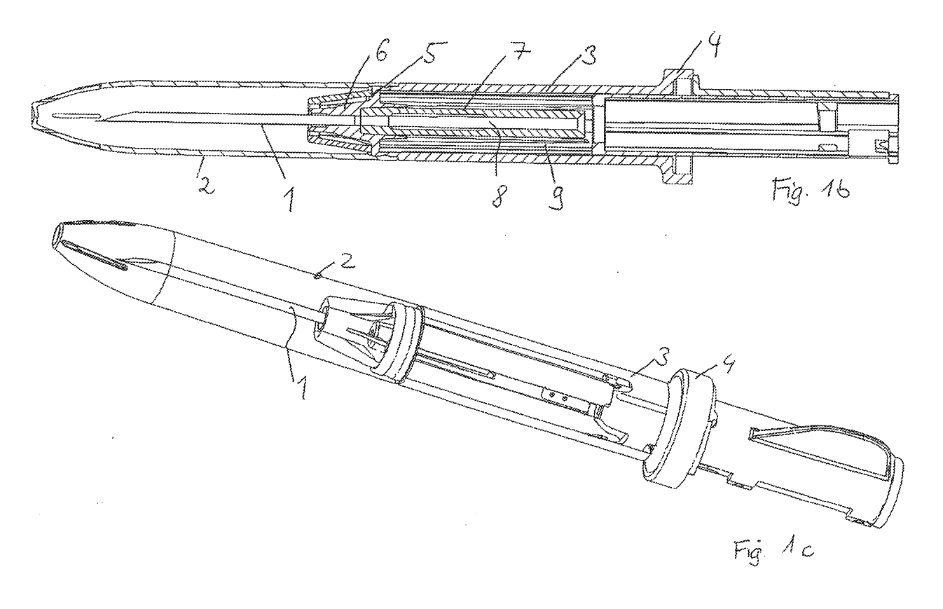

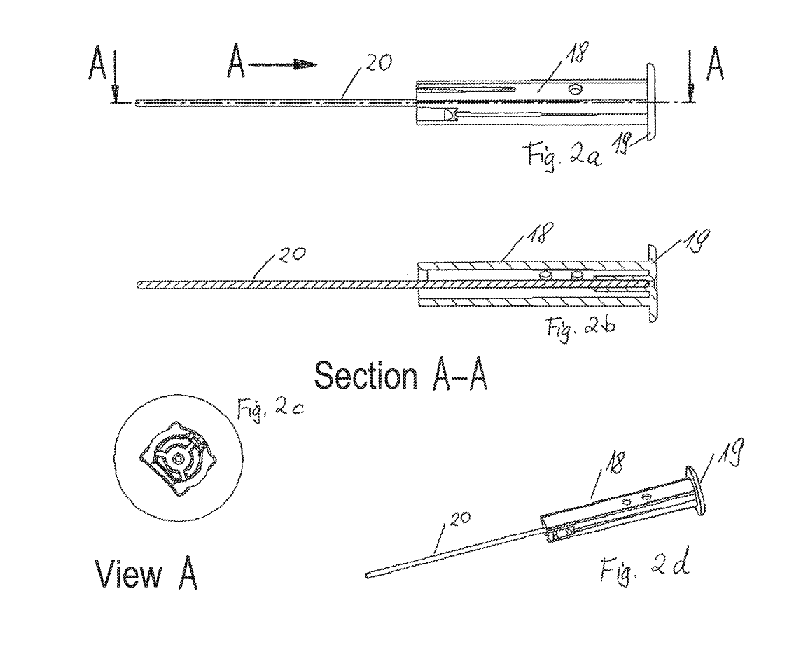

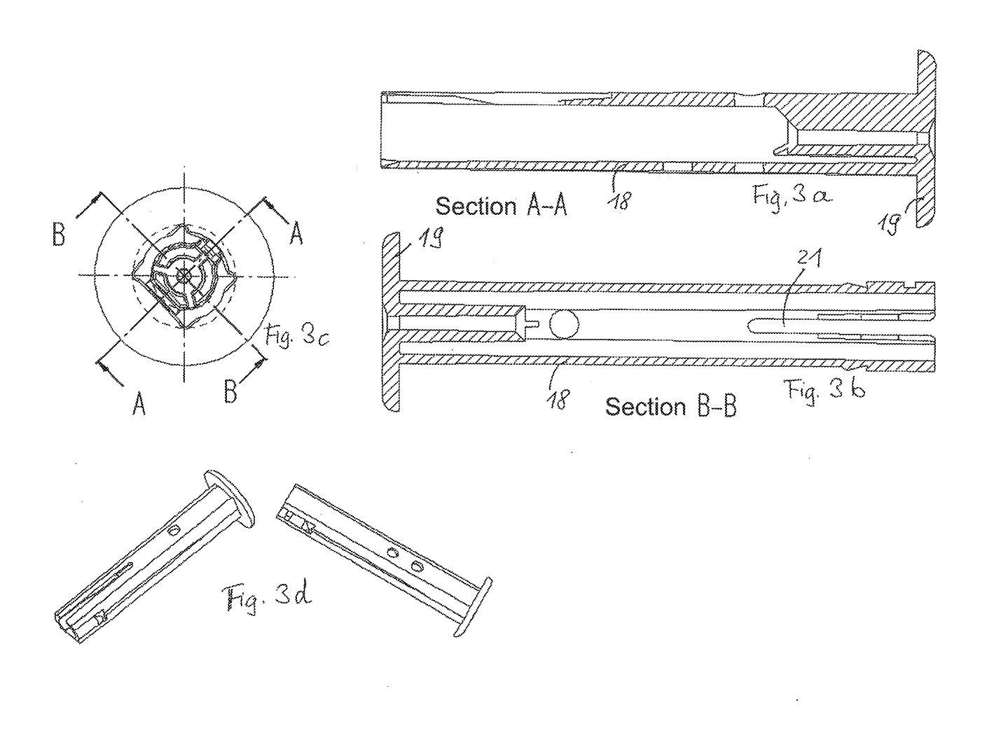

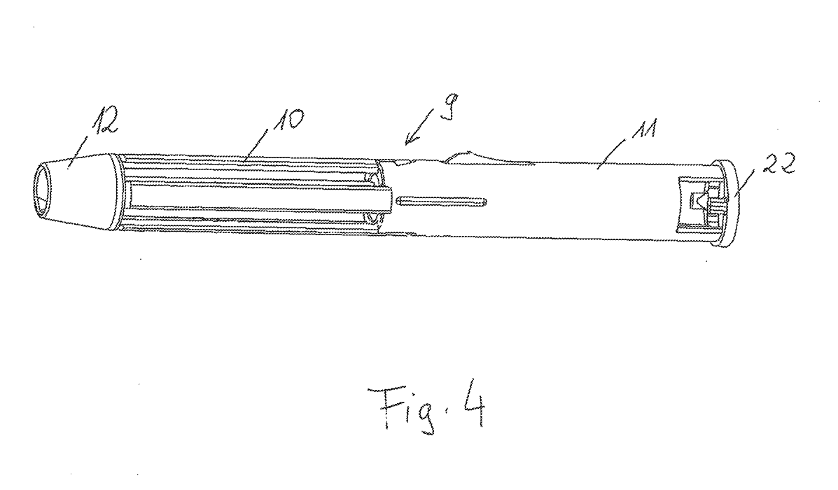

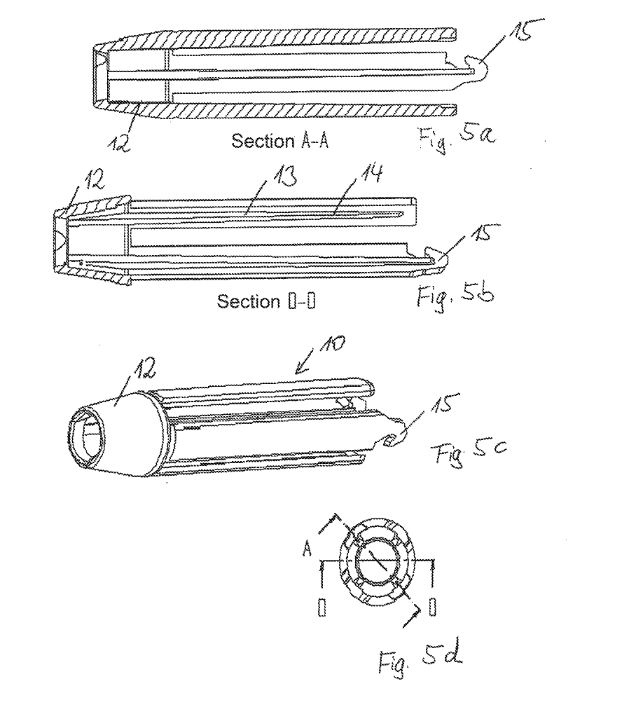

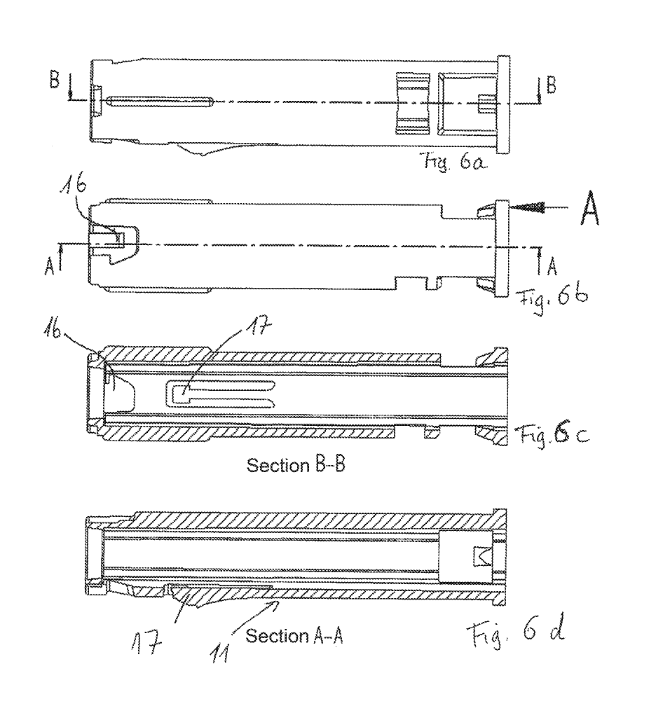

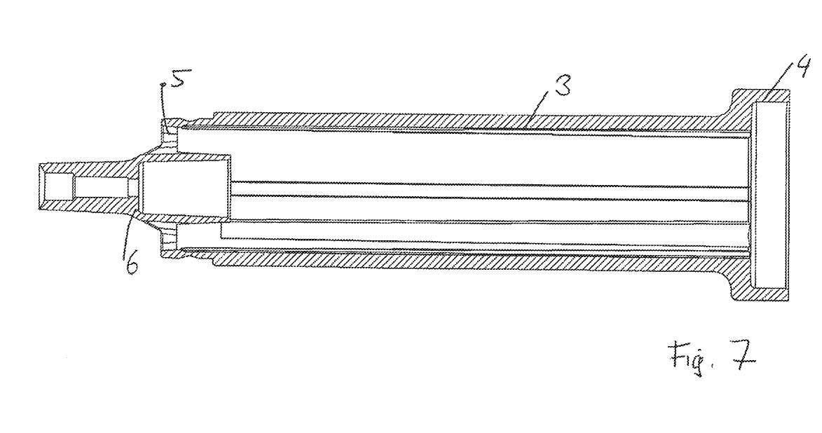

FIGS. 1a to 1c show an initial state of the implant syringe, in which the syringe needle 1 is covered by a removable protective cap 2. The removable protective cap 2 is readily removably snap connected to an outer sleeve 3. The outer sleeve 3 has an elongate cylindrical shape, wherein a radially projecting front gripping section 4 is integrally formed at its end section on the right in the figures. The outer sleeve 3 is firmly connected by means of a plurality of radially inwardly extending webs 5 to a needle holder 6, in which the syringe needle 1 is fastened. A preparation receptacle 7 is so arranged axially behind the needle holder 6 that its passage 8 is in alignment with the passage of the syringe needle 1. Situated in this preparation receptacle 8 there is a preparation, which is not shown, which, when the implant syringe is used, is delivered e.g. into the abdominal wall of a patient.

The outer sleeve 3 sits (when it is not blocked) movably on an inner sleeve 9, which is composed of a front portion 10 and a rear portion 11. The front portion 10 includes a front section 12, which narrows frustoconically and which is closed in the peripheral direction and covers the tip of the completely retracted syringe needle. The axially adjoining region includes webs 13, which are spaced in the peripheral direction and are provided with slits 14, whereby formed on the end sections of two webs there are hooks 15, which latch into openings 16 in the rear portion 11 of the inner sleeve 9 in order to connect the two portions.

The rear portion 11 of the inner sleeve 9 includes a spring arm 17, which is cut free from the wall of the inner sleeve portion 11 and projects radially outwardly with a locking hook 25. This spring arm 17 with the locking hook 25 serves as a blocking device for the outer sleeve 3 so long as a rear sleeve 18 is not slid into its end position in the rear portion 11 of the inner sleeve 9.

Integrally formed at the rear end of the rear sleeve 18 there is a flat gripping member 19, secured centrally to which in the sleeve 18 there is a rod-shaped piston 20. The rear sleeve 18 includes a slot 21 extending in the axial direction, which, when the rear sleeve 18 is slid into the rear portion 11 of the inner sleeve 9, so cooperates with the spring arm 17 by means of an oblique surface 26 that its locking hook 25 is drawn radially inwards. This is the case when the inner surface of the rear gripping member 19 abuts the rear edge 22 of the inner sleeve 9, whereby this position is readily releasably locked.

The outer sleeve 3 can now be retracted together with the injection needle 1 in its end position, in which the two gripping members 4 and 19 engage one another. The tip of the injection needle 1 is now situated within the frustoconical section 12 of the inner sleeve 9.

FIG. 1a shows a safety sleeve 22, which, in the initial state of the implant syringe, blocks the advance of the rear sleeve 18 and thus of the piston 12 with webs. The safety sleeve 22 is removed before use of the implant syringe.

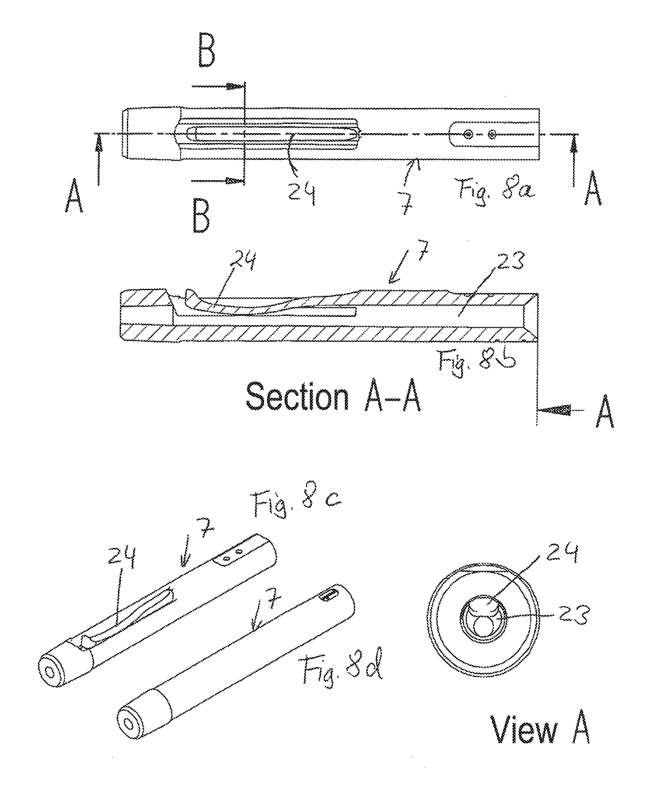

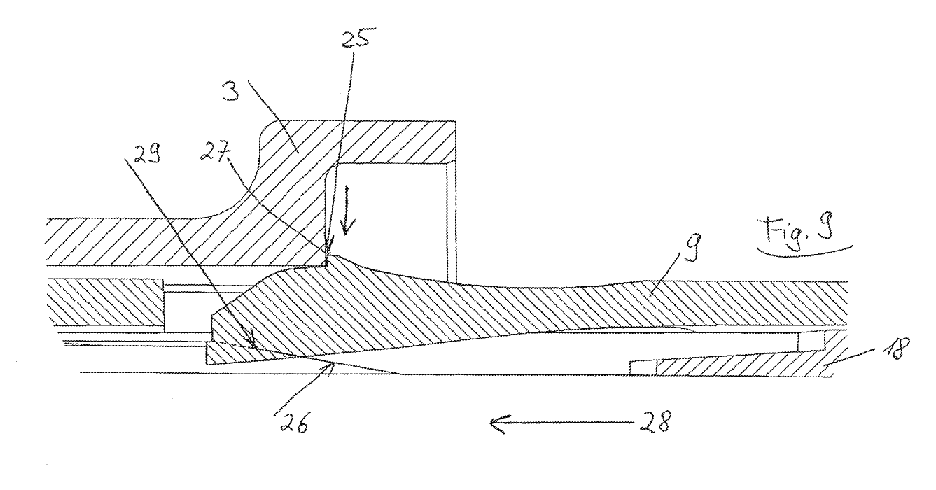

FIGS. 8a to 8d show the preparation receptacle 7, which is arranged behind the syringe needle holder. Projecting into the through passage 23 of the preparation receptacle 7 there is a spring arm 24, which is cut free from the peripheral wall of the preparation receptacle 7 and which, in the unloaded state, so constricts the through passage 23 that a preparation received therein cannot pass the spring arm 24. The spring arm has a gently convex shape projecting into the passage 23 and is constructed integrally with the remainder of the preparation receptacle 7. When the piston 20 is advanced, it presses the preparation smoothly through the constriction, whereby the spring arm is pressed somewhat upwardly. The preparation can thus not be damaged.

FIG. 9 is an enlarged schematic view of the blocking device for the outer sleeve 3. The inner sleeve 9 engages a rear annular shoulder 27 of the outer sleeve in this view with a radially outwardly projecting locking hook 25. When the rear sleeve 18 is advanced in the direction of the arrow 28, an oblique surface 26 on the rear sleeve 18 slides along a release ramp 29 on the inner sleeve 9, whereby the locking hook 25 is pulled radially inwardly and permits the outer sleeve 3 to be pulled back.

1. An implant syringe comprises a syringe needle holder, which holds an injection needle, an axially adjoining preparation receptacle and an outer sleeve with a radially projecting front gripping section on its rear end section, wherein the injection needle holder and the outer sleeve are connected together for common axial movement, including an inner sleeve, on which the outer sleeve is slidably located and in which the syringe needle is movable with the preparation receptacle, wherein the inner sleeve has axial slots, through which webs extend, which connect the outer sleeve to the syringe needle holder, further including a rod-shaped piston, which is fastened to a rear gripping section and is partially surrounded by a rear sleeve, which movably engages in the inner sleeve, wherein the rod-shaped piston is advanceable through the preparation receptacle into the syringe needle so far that a spacing remains between the tip of the syringe needle and the head end of the piston, which is approximately equal to the length of the preparation to be delivered, and the advance of the piston is limited by the rear gripping section reaching the rear edge of the inner sleeve, wherein the outer sleeve is releasably locked in the advanced starting position with the syringe needle exposed by a blocking device, which is arranged on the inner sleeve and projects radially outwards, and wherein the blocking device is moved radially inwards on advance of the rear sleeve into its end position, whereby the outer sleeve is released and is retractable by means of the front gripping section on the inner sleeve.

2. An implant syringe as claimed in claim 1, wherein the blocking device is a spring arm cut free from the inner sleeve, the head end of which, which is preferably shaped in the form of a locking hook, projects radially beyond the inner sleeve in the unloaded state and blocks the retraction of the outer sleeve.

3. An implant syringe as claimed in claim 1, wherein the rear sleeve has an axial slot, which slides over the spring arm, which is thus drawn radially inwardly by at least one oblique surface.

4. An implant syringe as claimed in claim 1, wherein the front end position of the rear sleeve is reached when the rear gripping section abuts the rear edge of the inner sleeve.

5. An implant syringe as claimed in claim 1, wherein the retraction of the outer sleeve is limited by the two gripping sections engaging one another.

6. An implant syringe as claimed in claim 1, wherein when the outer sleeve is completely retracted, the piston projects beyond the tip of the syringe needle.

7. An implant syringe as claimed in claim 1, wherein projecting into the passage of the preparation receptacle there is a spring arm which, in the unloaded state, blocks the movement of the preparation.

8. An implant syringe as claimed in claim 7, wherein the spring arm projects in a flatly convex curved manner into the passage.

9. An implant syringe as claimed in claim 1, wherein the inner sleeve includes a head section, which adjoins the slotted region and is closed over the periphery and which covers the tip of the syringe needle in its retracted end position.

10. An implant syringe as claimed in claim 9, wherein the head section has a forwardly tapering frustoconical shape.
